(19) United States
(12) Patent Application Publication
(10) Pub. No.: US 2005/0164368 A1
(43) Pub. Date: Jul. 28, 2005

(54) Bifidobacterium longum AR81 (KCCM-10492) enabling inhibition of rotavirus and active protein separated therefrom

(76) Inventors: Geun Eog Ji, Seoul (KR); Myeong Soo Park, Seoul (KR); Dong-Hyun Kim, Seoul (KR); Eun Ah Bae, Seoul (KR)

Correspondence Address: FISH & RICHARDSON PC, 225 FRANKLIN ST, BOSTON, MA, 02110, US

(21) Appl. No.: 10/938,162

(22) Filed: Sep. 10, 2004

(30) Foreign Application Priority Data
Sep. 26, 2003 (KR) 2003-67066

(51) Int. Cl. A61K 45/00; C12N 9/00; C12N 1/20

(52) U.S. Cl. 435/252.9; 424/93.45; 435/183

(57) ABSTRACT

The present invention relates to a novel strain enabling inhibition of rotavirus that is known as a principal cause of acute diarrhea and enteritis in children and in the young animals of cattle, horses, pigs and monkeys, etc, and an active protein also having an anti-rotavirus activity produced from the same.

The novel strain of the present invention was named as Bifidobacterium longum AR81 (KCCM-10492). The novel strain of the present invention and the active protein separated from the same can be effectively used for the production of medicine for intestinal disorders and especially for the production of baby goods.

### BACKGROUND OF THE INVENTION

(a) Field of the Invention

The present invention relates to a novel strain having an activity of inhibiting rotavirus and an active protein separated therefrom, more precisely, a novel strain Bifidobacterium longum AR81 (KCCM-10492) and an active protein also having an anti-rotavirus activity produced from the same.

(b) Description of the Related Art

A particle of rotavirus shape was found by Ms. Bishop, et al. (Australia) in 1973 in biopsy sample of duodenum of a child hospitalized for the treatment of acute diarrhea.

Rotavirus belongs to Family Reoviridae and was given the name ‘rota’ because its double capsid looks like a wheel of the cart. Ever since it was first found, rotavirus has been confirmed by numbers of research teams in the entire world to be a major cause of acute diarrhea in babies.

Then, rotavirus was separated by Light and Hodes in 1943 in feces of a baby having gastroenteritis. And the morphological characteristics of the virus were disclosed under electron microscope in 1977. In the meantime, reovirus-like virus was isolated from mammals and birds, and the virus was later confirmed to be rotavirus. So, the virus, rotavirus, was finally grouped into Genus Rotavirus belonging to Family Reoviridae.

Rotavirus is divided into 6 different antigen groups (A-f). Rotaviruses of group A, B and C are found in both human and animals, and rotaviruses of group D, E and F are found in animals only. Groups divided by serological characteristics have a common or a cross antigen. Rotavirus of group A has been studied most since it was first reported, so that its characteristics have been investigated thoroughly. Rotavirus of group A is largely distributed among human-derived strains, and subgroups are determined by its inner capsid protein VP6 and serological types are determined by outside capsid protein VP7 and VP4. At least two subgroups and four serological types have been disclosed so far.

Rotavirus has an outside capsid and the core without envelope. The viral genome is composed of 11 individual segments of double helical RNA. Outside capsid of the virus is composed of two proteins ‘VP4 and VP7’. VP4, a cell adherent protein and a hemagglutinin, stands out like a spike from the surface of the virus and takes 2.5% of the total weight of the virus. VP4 is split into VP5* (Mᵣ 60,000) and VP8* (Mᵣ 28,000) to penetrate into inner cells. VP7 is a glycoprotein having the weight of 37 kDa, taking 30% of the total weight of the virus, and constructs cellular shell of the smooth outside capsid.

Table 1 below presents general characters of rotavirus.

TABLE 1

| | |
|---|---|
| Virion: | Icosahedral |
| Diameter: | 60-80 nm |
| | Double capsid shell |
| Composition: | RNA (15%), protein (85%) |
| Genome: | 11 segments composed of double helical RNA |
| Total molecular weight: | 12-15 million Dalton |
| Protein: | 9 structural proteins |
| | Core includes a couple of enzymes |
| Envelope: | No |
| Replication: | Cytoplasmic replication |

Rotavirus is known as a principal cause of acute diarrhea and enteritis in children and in the young animals of cattle, horses, pigs and monkeys. An infection route of the virus is from feces through the mouth. Rotavirus causes an infectious acute diarrhea in babies under age 2, which is so called infantile gastroenteritis or acute gastroenteritis.

Diarrhea is a result of troubles in absorption of intestinal mucosal epithelial cells caused by the infection with rotavirus. Once villus cells of small intestine are infected with the virus, the virus proliferates in cytoplasm of those cells to cause troubles in transport system. Damaged cells are apart and spout out the virus in intestinal track, so that the virus can be found in feces. When rotavirus infiltrates, damaged cells of villi are replaced by immature crypt cells not having absorption capability, causing malfunction of absorption of sodium and glucose, resulted in diarrhea.

Rotavirus induced diarrhea in babies mostly comes out briefly right after the beginning of weaning, which is called “Weanling diarrhea” or “Colibacillosis”. Such diarrhea, especially in an aerobic strain, is inevitably accompanied with the transition to non-hemolytic E. coli and hemolytic E. coli of Enterococci. Hemolytic E. coli generates enterotoxin and harbors pili which enables E. coli to adhere to an enterocyte. Such strains are named enteropathogenic E. coli or enterotoxigenic E. coli, and have various serotypes.

It was reported in 1973 that a very serious diarrhea was developed in a piglet bred without colostrum. After investigating the cause of the disease, it was diagnosed as colibacillosis. The serious diarrhea in a piglet that was bred without colostrum was caused by the infection with diarrheal fluid harboring retrovirus destroying cells by infiltrating in enterocytes. And numbers of retrovirus were found in diarrheal feces of a three to four week old piglet in weaning period. It was important to find out the cause of diarrhea in a piglet and rotavirus was believed to be a relevant principal cause of the disease. It was also confirmed that non-hemolytic E. coli was transformed into hemolytic E. coli during diarrhea. However, when hemolytic and enteropathogenic E. coli were orally administered without rotavirus to a piglet, diarrhea didn't come out. In conclusion, rotavirus damages intestinal track to builds up a proper environment for enteropathogenic E. coli to move into small intestines and form a colony.

Clinical symptoms of a retrovirus induced disease are vomiting, diarrhea, abdominal pain and fever, which are mostly related to intestinal tracks. In particular, dehydration and electrolyte imbalance are progressed fast in babies.

According to the statistical data of World Health Organization (WHO), 5 million children under age 5 on earth die of diarrhea every year, and 20% (one million) of them died of rotavirus induced diarrhea. According to a report that investigated more than 12 possible causes of diarrhea for 12 months with children in Seoul, rotavirus was detected in 47% of children suffering from diarrhea, which was the highest appearance exceeding statistics of WHO (20%). Rotavirus induced diarrhea prevails in winter and is the leading cause of infant mortality in the developing countries. The best precaution to prevent the disease is breast-feeding. Since mother's milk includes IgA acting against rotavirus, infantile diarrhea caused by rotavirus can be prevented by breast-feeding.

Table 2 below presents the statistics about enteropathogenics of Korean children with and without diarrhea.

TABLE 2

No. (%) of confirmed
enteropathogenic^{a}

| Enteropathogenic | Diarrhea group^{b} | Control group^{c} |
|---|---|---|
| Rotavirus | 107(46.5) | N.D. |
| Enterotoxigenic E. coli | 52(22.5) | 14(13.5) |
| (ETEC) | | |
| Other ST | 9(4.8) | 1(1.0) |
| Enterobacteriaceae | | |
| Enteroadherent E. coli | 34(14.7) | 8(7.7) |
| (EAEC) | | |
| Enteropathogenic E. coli | 15(6.5) | 5(4.8) |
| (EPEC) | | |
| Shigella spp. | 4(1.7) | 0 |
| Salmonella spp. | 1(0.4) | 0 |
| Campylobacter jejuni | 1(0.4) | 0 |
| Enteroinvasive E. coli | 1(0.4) | 0 |
| (EIEC) | | |

^{a}Numbers of pathogens separated from one patient were all recorded.
^{b}n = 231
^{c}n = 104

56 patients (24.2%) with diarrhea were confirmed not to have a pathogen, and 52 people (52.9%) in a control group were confirmed not to have a pathogen.

Most patients are well recovered by the supply with water, but it is not easy for patients of developing countries to be treated simply with water. Although the frequency of development of bacterial diarrhea decreases by the supply with fresh water and sanitation, rotavirus induced diarrhea does not much decreased.

Infection with rotavirus causes the synthesis of IgA secreted in lumen. According to the experiments with sheep, calves and pigs, an antibody secreted in lumen is more important to prevent rotavirus infection than any other antibody existing in serum or lymph.

Thus, it is preferred to develop a live virus vaccine for oral administration, and thus, attempts have been made to develop a subunit vaccine or a microencapsulated killed vaccine.

Every effort has been made for the past 20 years to develop a novel vaccine and both conventional and molecular biological methods have been tried so far. Nevertheless, the development of a novel vaccine without much side effect was not successful. A vaccine was once developed, but it was not good enough for the use because of serious side effects.

### SUMMARY OF THE INVENTION

The present inventors paid attention to the point that rotavirus infection shows up largely in the weaning period, during which the number of Bifidobacterium residing in intestines decreases rapidly but the number of E. coli increases.

And so, it is an object of the present invention to provide a novel Bifidobacterium strain ‘Bifidobacterium longum AR81 (KCCM-10492)’ having an activity of inhibiting rotavirus infection, in order to solve the above-mentioned problem.

It is another object of the present invention to provide an active protein having an anti-rotavirus activity produced from the strain of the invention.

### BRIEF DESCRIPTION OF THE DRAWINGS

FIG. 1 is a schematic diagram showing the separation and the purification processes of the active protein of the present invention.

### DETAILED DESCRIPTION OF THE PREFERRED EMBODIMENTS

The present invention provides a novel Bifidobacterium strain ‘Bifidobacterium longum AR81 (KCCM-10492)’ having an activity of inhibiting rotavirus and a protein acting against rotavirus which is produced from the same.

The novel Bifidobacterium strain inhibiting rotavirus of the present invention was named ‘Bifidobacterium longum AR81 (KCCM-10492) and was deposited at Korean Culture Center of Microorganisms (KCCM) on May 6, 2003 (Accession No: KCCM-10492).

The characteristics of Bifidobacterium longum AR81 (KCCM-10492) isolated by the present inventors are shown in below Table 3.

TABLE 3
Characteristics of Bifidobacterium longum AR81

| Morphology | Rods or Y-shaped |
|---|---|
| Gram staining | + |
| F6PPK | + |

Protein acting against rotavirus was separated from the strain and a nucleotide sequence of a gene coding the protein was represented by SEQ. ID. No 1. Amino acid sequence thereof was also represented by SEQ. ID. No 2.

The protein of the present invention was confirmed to act against rotavirus. A pharmaceutical composition containing the protein of the present invention can include pharmaceutically acceptable excipients, carriers, diluents, etc., and can be produced in the forms of tablets, pills, disperse preparation, granules, elixirs, suspensions, emulsions, solutions, syrups, etc. The composition can be administered either orally or parenterally and its dosage of a day is 1-20 mg/kg weight.

For the separation and purification of the protein, the novel strain of the invention ‘Bifidobacterium longum AR81 (KCCM-10492) was cultured in a trypic soy broth (pH 7.2) supplemented with 0.1% ascorbic acid and 0.01% sodium thioglycollic acid, followed by centrifugation, precipitation and dialysis.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

Target Cells and Culture of the Cells

The target cell line used in the present invention was Macaccus' Rhesus monkey kidney cell line (MA-104), which was provided from Toyama sanitary testing and research institute, Japan.

MA-104 cells were cultured in a 37° C. 5% CO₂ incubator using DMEM supplemented with 10% FBS, 1% antibiotics-antifungal agent and 3.5 g/L of sodium bicarbonate.

### EXAMPLE 2

Distribution of Rotavirus and Preparation of the Virus Solution

SA11 rotavirus was provided from National Institute of Health (NIH), Korea and Wa rotavirus was provided from ATCC.

Each virus was distributed into T.C. flasks (25 cm²) by the concentration of 1-2×10⁶ cells/flask, then cells were adhered for one hour under the condition of 37° C. and 5% CO₂.

Supernatant was removed and the cells were washed with FBS-free DMEM medium. 20 μl of DMEM medium (infectious medium) containing 5 μg/ml of trypsin was added to 400 μl of Wa virus or SA11 virus solution, which was treated at 37° C. for 30 minutes. 400 μl of pre-activated Wa virus solution was spread over the cell surface evenly, leading to infection at 37° C. for one hour.

Upon completing the infection, supernatant was removed and cytopathic effect (cpe) was investigated. After confirmation, it was frozen.

Freezing-thawing was repeated three times to break the cell membrane completely. Centrifugation was performed at 4° C., 500 rpm for 20 minutes. Supernatant, a virus solution, was taken only and kept in a refrigerator.

### EXAMPLE 3

Lactobacillus and Bifidobacterium used in the Present Invention

Lactobacillus acidophilus KCTC 3150, Bifidobacterium longum KCTC 3215 and Bifidobacterium infantis KCTC 3226 used in the present invention were provided from Institute of Genetic Engineering, Korea, and the rest of the strains used in the invention were separated from bacterial flora residing in the human intestines.

### EXAMPLE 4

Determination of Titer of Rotavirus Solution

(End-Point Dilution Method)

MA-104 cells cultured in the above Example 1 were treated with 0.25% trypsin-EDTA to isolate them. Then, a fresh medium was provided. Centrifugation was performed at 1200 rpm for 5 minutes to discard supernatant. Cell concentration was adjusted to 5×10⁵ cells/ml using infectious medium.

Rotavirus stock solution was serially diluted from 10⁻¹ to 10⁻⁸, and each diluted solution was distributed to 8 wells by 100 μl.

The cultured MA-104 cells were distributed thereto by the same amount, followed by further culture in a 37° C., 5% CO₂ incubator for 7 days.

Upon completing the culture, cpe of each well was investigated under inverted microscope to calculate TCID₅₀ (50% Tissue-culture infectious dose)/ml. Pfu (plaque forming unit)/ml was calculated by multiplying the value of TCID₅₀/ml by a coefficient 0.69.

### EXAMPLE 5

The Effect of Rotavirus Infection on Components of Lactobacillus Cytoplasm

Each strain used for the experiments was cultured in 5 ml of GAM broth for 18 hours. Centrifugation was performed at 3000 rpm for 20 minutes, resulting in the preparation of bacteria precipitate. The precipitate was suspended in 1 ml of PBS, followed by sonication. Centrifugation was then performed at 12000 rpm for 60 minutes to obtain supernatant, resulting in lactobacillus cytoplasm solution.

The prepared cytoplasm solution was filtered with 0.45 μm syringe filter for sterilization.

The cytoplasm solution of each strain was diluted to the concentrations of 0.01, 0.02 and 0.04 mg protein/ml, which was added by 50 μl to each well containing MA-104 cells and SA11 virus or Wa virus diluted solution, followed by further culture. Then, cpe was investigated and the results were shown in Table 4 and Table 5.

TABLE 4

| | Infection inhibition rate (%) | | |
|---|---|---|---|
| | Cytoplasmic component (mg/ml) | | |
| Bifidobacterium | 0.0005 | 0.002 | 0.010 |
| B-47 | 16.7 | 33.3 | 66.6 |
| B-81 | 0 | 33.3 | 100 |
| B-180 | 0 | 16.7 | 83.3 |
| KK-11 | 0 | 16.7 | 66.6 |
| KK-12 | 0 | 16.7 | 83.3 |
| B. longum^{a} | 0 | 14.3 | 14.3 |
| B. infantis^{a} | 0 | 28.6 | 42.9 |
| L. acidophilus^{a} | 0 | 14.3 | 28.6 |

B-81: Bifidobacterium longum AR81 (KCCM-10492)
^{a}KCTC

TABLE 5

| | Infection inhibition rate (%) | | |
|---|---|---|---|
| | Cytoplasmic component (mg/ml) | | |
| Bifidobacterium | 0.0005 | 0.002 | 0.010 |
| B-47 | 16.7 | 16.7 | 66.6 |
| B-81 | 0 | 33.3 | 100 |
| B-179 | 0 | 33.3 | 83.3 |
| KK-11 | 0 | 16.7 | 66.6 |
| KK-12 | 0 | 16.7 | 83.3 |
| B. longum^{a} | 0 | 14.3 | 14.3 |
| B. infantis^{a} | 0 | 28.6 | 42.9 |
| L. acidophilus^{a} | 0 | 14.3 | 42.9 |

B-81: Bifidobacterium longum AR81 (KCCM-10492)
^{a}KCTC

Among lactic acid bacteria, cytoplasmic components of Bifidobacterium animalis KCTC 3126 and 10 Bifidobacterium infantis KCTC 3226 were confirmed to have very good infection inhibition effect. Among isolated strains, 47, 81 (Bifidobacterium longum AR81 (KCCM-10492)) and 179 strains showed excellent inhibition effect, and in particular, strain 81, a novel strain of the present invention, showed the best inhibition effect.

Isolated strains, strain 47, 81 and 179, were confirmed to be Gram-positive. And their morphological characteristics are equal to those of Bifidobacteria (rods shaped, Y-shaped, etc.). After investigating the activity to Bifidobacteria-specific enzyme F6PPK (fructose 6 phosphate phosphoketolase), the above strains were all confirmed to be positive, indicating that they are all Bifidobacteria.

Among isolated strains, highly activated strain 47 and strain 81 were identified by 16S rDNA nucleotide sequence analysis. In order to identify a strain using 16S rDNA sequence, PCR primer had to be prepared first. The general 16S rDNA sequence of Bifidobacterium was obtained from Gene Bank, whose size was about 1.5 kb and preservative sequence was found in inside. Based on that sequence, 4 primers were constructed.

A template for PCR was prepared by using Genereleaser kit. Then, 0.5 kb long DNA fragment was prepared by using primers 616V and 610R in order to obtain a sequencing template. Then, 0.5 kb DNA was obtained by using 612F and 630RIII.

Electrophoresis was performed to recover DNA band using a gel extraction kit (Qiagen, U.S.A). TA cloning to pGEM T easy vector (Promega, USA) was performed for sequence confirmation.

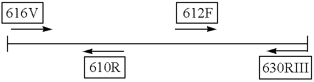

After comparing the sequences of strain 47 and strain 81 with others of Gene Bank, those sequences were confirmed to have 99% homology with that of Bifidobacterium longum, leading to the identification of them as B. longum. In particular, strain 81 was named ‘Bifidobacterium longum AR81 (KCM-10492)’ and deposited at Korean Culture Center of Microorganisms (KCCM) on May 6, 2003.

Below are the information about primers to strain 47 and strain 81, a novel strain of the present invention.

Strain 47 (forward primer):
1. Bifidobacterium longum NCC2705 section 172 of 202 of the complete genome Length=10301 Score=1061 bits (535), Expect=0.0 Identities=553/559 (98%), Gaps=1/559 (0%) Strand=Plus/Plus

Strain 47 (reverse primer)
1. Bifidobacterium longum NCC2705 section 172 of 202 of the complete genome Length=10301 Score=940 bits (474), Expect=0.0 Identities=477/478 (99%) Strand=Plus/Plus

Strain 81 (forward primer):
1. Bifidobacterium longum NCC2705 section 172 of 202 of the complete genome Length=10301 Score=1061 bits (487), Expect=0.0 Identities=486/487 (99%), Gaps=1/487 (0%) Strand=Plus/Plus

Strain 81 (reverse primer)
1. Bifidobacterium longum NCC2705 section 172 of 202 of the complete genome Length=10301 Score=1061 bits (521), Expect=0.0 Identities=520/521 (99%), Gaps=1/521(0%) Strand=Plus/Plus

### EXAMPLE 6

The Effect of Rotavirus Infection on the Components of Lactobacillus Cell Wall

The strains used in the experiments were cultured in 5 ml of GAM broth for 18 hours. Then, centrifugation was performed at 3000 rpm for 20 minutes, resulting in the preparation of bacteria precipitate. The precipitate was suspended in 1 ml of PBS, followed by sonication. Centrifugation was performed again at 12000 rpm for 60 minutes to obtain precipitate, which was prepared as lactobacillus cell membrane and cell wall. The precipitate was suspended in 1 ml of PBS, to which the same amount of ether was added. The solution was left for 24 hours for sterilization.

The cytoplasmic solution of each strain was diluted to make final concentration to be 0.02, 0.04 and 0.08 mg protein/ml each, which was added by 50 μl to each well containing the diluted MA-104 cells and SA11 virus or Wa virus solution. After culturing, cpe was investigated, and the results are shown in Table 6 and Table 7.

TABLE 6

| | Infection inhibition rate (%) | | |
|---|---|---|---|
| | Cell wall (mg/ml) | | |
| Bifidobacterium | 0.0005 | 0.002 | 0.010 |
| B-47 | 16.7 | 0 | 16.7 |
| B-81 | 0 | 28.6 | 16.7 |
| B-180 | 0 | 16.7 | 33.3 |
| KK-11 | 16.7 | 0 | 16.7 |
| KK-12 | 16.7 | 0 | 16.7 |
| B. longum^{a} | 0 | 14.3 | 0 |
| B. infantis^{a} | 0 | 14.3 | 0 |
| L. acidophilus^{a} | 0 | 0 | 0 |

B-81: Bifidobacterium longum AR81 (KCCM-10492)
^{a}KCTC

TABLE 7

| | Infection inhibition rate (%) | | |
|---|---|---|---|
| | Cell wall (mg/ml) | | |
| Bifidobacterium | 0.0005 | 0.002 | 0.010 |
| B-47 | 0 | 0 | 16.7 |
| B-81 | 0 | 28.6 | 16.7 |
| B-179 | 0 | 16.7 | 33.3 |
| KK-11 | 16.7 | 16.7 | 16.7 |
| KK-12 | 16.7 | 16.7 | 16.7 |
| B. longum^{a} | 0 | 14.3 | 14.3 |
| B. infantis^{a} | 0 | 14.3 | 14.3 |
| L. acidophilus^{a} | 14.3 | 14.3 | 14.3 |

B-81: Bifidobacterium longum AR81 (KCCM-10492)
^{a}KCTC

As shown in Table 6 and Table 7, a cytoplasmic component of lactobacillus is better at inhibiting virus infection than a cell wall component.

### EXAMPLE 7

Characteristics and Determination of Wa Virus Infection Inhibitor Included in Lactobacillus

Lactobacillus was cultured in GAM broth. Cells were collected by centrifugation, which were suspended in physiological saline, followed by sonication. Centrifugation was performed again to obtain supernatant. The supernatant was autoclaved, filtered, heat-treated (100° C., 15 minutes), incinerated or precipitated with acetone.

The precipitate was diluted to make the final concentrations (V/V %) of 5, 10 and 20%, which were added by 50 μl to each well containing MA-104 cells and diluted rotavirus solution. Then cpe was investigated. The results are shown in Table 8.

TABLE 8

| | | Inhibition rate |
|---|---|---|
| Lactobacillus | Treatment | (%) |
| B-47 | Autoclaving | 0 |
| | Heating | 0 |
| | Filtration | 66.6 |
| | Incineration | 0 |
| | Acetone precipitation | 50 |
| B-81 | Autoclaving | 16.7 |
| | Heating | 0 |
| | Filtration | 50 |
| | Incineration | 0 |
| | Acetone precipitation | 33.3 |
| KK-11 | Autoclaving | 0 |
| | Heating | 0 |
| | Filtration | 50 |
| | Incineration | 0 |
| | Acetone precipitation | 33.3 |

^{a}Final concentration (%)
^{b}Not detected

While virus infection inhibiting effect of lactobacillus decreased or disappeared by autoclaving, heating, incineration, etc., the effect slightly decreased by acetone precipitation and was sustained by filtering for sterilization. Thus, it was judged that a protein was the substance inhibiting Wa virus infection.

### EXAMPLE 8

Cloning of a Gene of a Protein Having an Anti-Rotavirus Activity

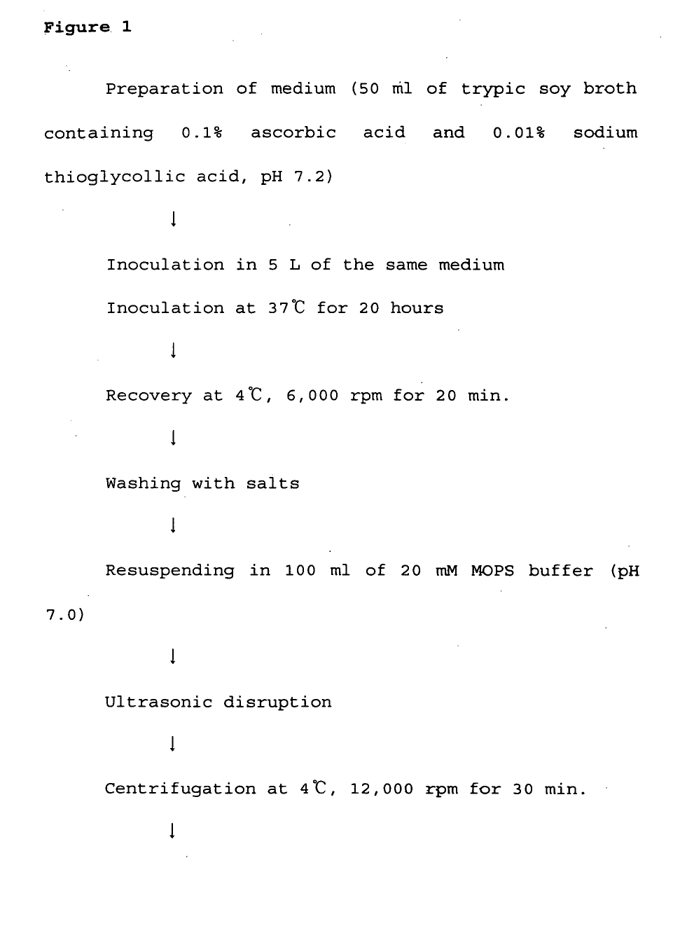

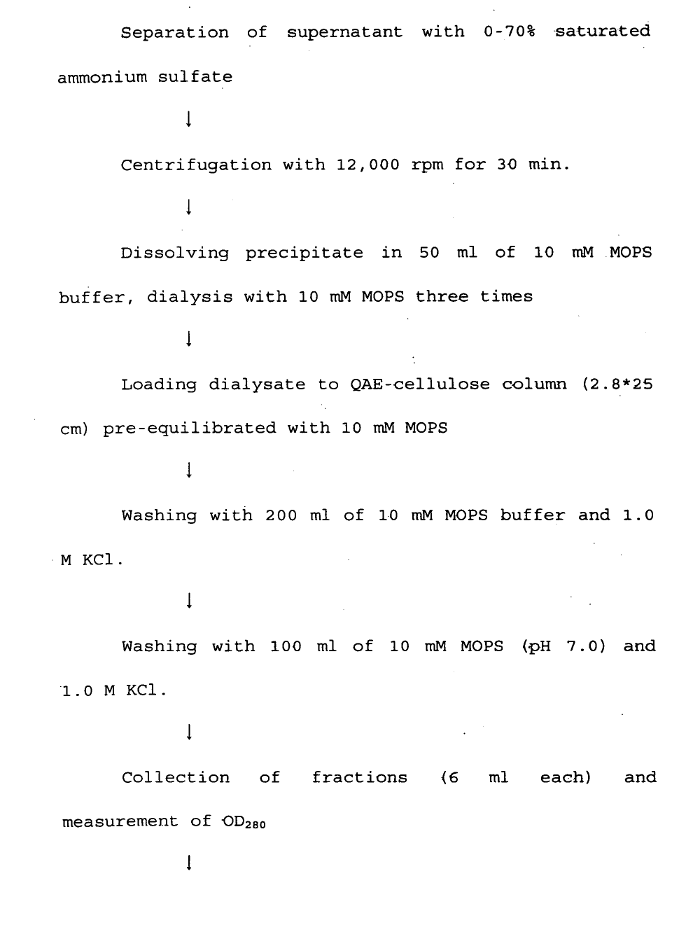

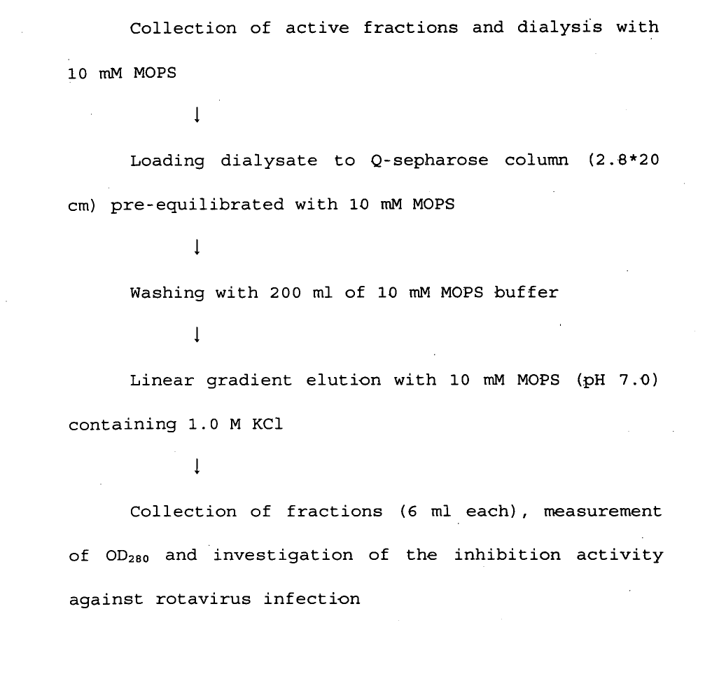

For the cloning of a gene of a protein having an anti-rotavirus activity, a protein was separated and purified from selected strains by the processes described in FIG. 1.

### EXAMPLE 9

Identification of an Amino Acid Sequence of an Active Protein Having an Anti-Rotavirus Activity

Internal sequence assay was performed to identify an amino acid sequence of the protein, separated/purified in the above Example 8, having an anti-rotavirus activity.

The identification of an amino acid sequence was entrusted to Korea Basic Science Institute (KBSI), and as a result, an amino acid sequence composed of 10 amino acids was identified.
N′-HLDLAVENT-C′

After data base (GenBank) search, a corresponding sequence to the identified amino acid sequence was found in dipeptidaseD (GenBank Accession number NC_{—}004307), one of bifidus originated genes.

Based on that information, PCR was performed by using the below primers to amplify the region including a whole gene coding the above amino acid sequence.

| | |
|---|---|
| pepD-F: | 5′-AAAACCGATTGGCAACTCGGG-3′ |
| pepD-R: | 5′-GCCACTTCTGGGCGCCGGC-3′ |

Then, the PCR product was cloned into pGEM-Teasy vector to identify nucleotide sequence.

The nucleotide sequence is represented by SEQ. ID. No 1, and the protein thereof is represented by SEQ. ID. No 2.

### EXAMPLE 10

Expression of a Protein Having an Anti-Rotavirus Activity and Investigation of Titer Thereof.

In order to investigate an anti-rotavirus activity of the protein of Example 7, the expression of the protein was investigated by using E. Coli expression vector.

PCR was performed by using the below primers, and the product was linked to pBAD-TOPO.

| | |
|---|---|
| pepTOPO-F: | 5′-ATGGCCTGCACCACGATTCTGGTAGG-3′ |
| pepTOPO-R: | 5′-AAAGTCGGACATGTGGAAGCCGTTC-3′ |

E. coli was transfected with the vector. Then, the expression of the inserted gene was induced by using arabinose. Cells were recovered. Coenzyme solution was prepared by ultrasonic disruption. Then, an anti-rotavirus activity was investigated.

As shown in Table 9, an anti-rotavirus activity of the protein was higher than that of a control.

TABLE 9

Infection inhibition rate (%)

| Agent | 10X dilution | 100X dilution |
|---|---|---|
| Control | 25 | 0 |
| Transfected sample | 62.5 | 10 |

### EXAMPLE 1

Development of Powdered Medicine for Intestinal Disorders

Medicine for intestinal disorders for babies has been developed by using the novel strain of the present invention, which has an activity of inhibiting rotavirus.

First, the strain was inoculated to medium for bifidus production, followed by further culture (10 ml-->500 ml-->5 L-->50 L-->500 L-->5 KL) Centrifugation was performed to recover cells. The recovered cells were mixed with cryoprotectants, followed by quick-freeze and complete drying with a lyophilizer. Cells of lactobacillus for intestinal regulation were also recovered by the same processes as described above and then used for the production of medicine for intestinal disorders. In order to give intestinal regulating effect and other functional effects, the strain of the present invention can be mixed with galactooligosaccharides and other components according to the below composition.

TABLE 10

| Component | Content |
|---|---|
| Bifidus raw powder | 8% |
| Lactobacillus raw powder | 1% |
| Cyclodextrin | 28% |
| Galactooligosaccharide | 12% |
| Alpha Corn | 30.5% |
| Aloe Vera | 0.5% |
| Glycerine Fatty Acid Ester | 16% |
| Xilytole | 1% |
| Calcium Lactate | 3% |
| Lactoferrin concentrate | 0.05% |
| Vitamin B2 | 0.04% |
| Vitamin B12 | 0.00004% |
| Vitamin D3 | 0.00006% |

As explained hereinbefore, the novel Bifidobacterium strain of the present invention ‘Bifidobacterium longum AR81 (KCCM-10492)’ has an activity of inhibiting rotavirus that has been known as a principal cause of diarrhea in infants and in the young animals.

Particularly, the strain above and a protein having an anti-rotavirus activity separated from the same are useful for the production of medicine for intestinal disorders.

Moreover, the strain of the present invention and the protein separated therefrom can be used for the production of functional fermented milk for infants.

1. A novel strain Bifidobacterium longum AR81 (KCCM-10492) having an activity of inhibiting rotavirus.

2. A gene encoding a protein having an anti-rotavirus activity separated from the Bifidobacterium longum AR81 (KCCM-10492) of claim 1 and having a nucleotide sequence represented by SEQ. ID. No 1.

3. A protein having an anti-rotavirus activity separated from the Bifidobacterium longum AR81 (KCCM-10492) of claim 1 and having an amino acid sequence represented by SEQ. ID. No 2.

4. A medicine for intestinal disorders containing the novel strain of claim 1 or the protein of claim 3 as an effective ingredient.

5. The medicine for intestinal disorders as set forth in claim 4, wherein the form of the medicine is selected from a group consisting of liquid and powder.

6. The medicine for intestinal disorders as set forth in claim 5, wherein the medicine is for infants.


SEQUENCE LISTING (7 sequences)

SEQ ID NO: 1 - DNA, 3590 bp, Bifidobacterium longum
  CDS (971)...(2569)
aaaaccgattggcaactcgggaatatacaggcggcggcaccgatgcctctcacatcaccgggaaacgatgacggcaccggggaaaaccatcagcccacactgcggaaaaacactgccgaacctgtcgaacaaacattgaagcaaaacactcataacccgacgcaactagcccccctctgatgagtgggggctgtcgagcgtagcgagactgggggagagaccccgtaacgcacagcccccagtacgtcacacatatgagcgatactgggacgtatgaagatttccgccgatttcaccgtcatcccggacgatttcgccaaggccgcggcccctgaatatttcaatggtggcgtgcctgtggtctccttcccgttctacatcgatgatgtggatcccgaggcccgctacctgcactgggagttcgtggacccggattcgattccggtctgcggcttcgagtggattcactggtccgtggccaatctgccgattgatgcgctgatgtacgacttcaatgattcgcatgcgttggccatcccgccggacttctcccgtcagctgccggccatgattcccgagaccgtgcaggggcgcaactcctcggcctccaagttcttgggccgcagcactgacccggcggtgatcatgcggtacaacggtccgcagccgccggacaaggatcatgagtactacctgcaggtgtgggccaccaagaagccgttgcccggcttgaatcagggcttctggctcaacgagctgttgcacgcgctgcgcaacagcggtgaagtgcccgacaccgacggcatcttcctgaccggcaaggcgtgagccacatgagcccccgcaggcgggggctgtcagcatcactgactgggggtgggtcagtacaaaaccaccttcagtccgacttcgtcgtccagctcccgccagtgggagcgtgactacaacaaagaaaggacaccatcacactatggcctgcaccacgattctggtaggcaaagacgcgagctatgacggctcgaccatcatcgcccgcaacgaggattccgcgaacggcgagttcaacccgaagcgcttcatcgtcgtgaagccggaggaacagccgcgcgaatacaagtcggtgatctcccacctaaccatcaccctgccggacgacccgctgcagtacaccgccgtgccgaacgcggacctcaaggaaggtatttggggcgaggccggcgtcaacgaggcgaacgtggcgatgagcgccaccgaaaccctgaccaccaacgagcgcgtgctcggcgccgacccgttcgtggaatacacgccggccaagggcgatgagcccgaggtgcccggcggcatcggcgaagaggacttcctgaccatcgtgctgccgtatgtcaagaccgcacgcgagggcgtccagcgcctcggcgcgctgcttgaggaattcggcacctacgagatgaacggcgtcgccttctccgactccaacgaaatctggtggctggaaactgtgggcggccatcactggattgccaagcgcgtgccggacgaggcctacgtgaccatgccgaaccagctcggtatcgacgaattcgaccttgaggacgctctgggtgaccaggaagcgcacatgtgttccgaagaccttgccgaattcatcgaaaccaaccatttggacctcgcggtggagaacactaccccgttcaacccgcgcgatgccttcggctcccactctgactccgaccacgtgtacaacaccccgcgcgcctggtacatgcagcgcttcctcaacccgtacgacgaggtgtgggacggcccggacgccgaccacaagcccacctccgacgacatcccgtgggcccgtcagcctgagcgcaaggtgaccatcgaggatatcaagtacgtgctgagctcccactaccagggcaccccgttcgatccgtacggccagctgggcgatgagcgcacccgccacatgtaccgcacgatcggcatcaaccgccagagccagctcgcggtgatgcagatccgcccgtaccgcccgcaggcctcccgcgccatccagtggatggcatacggctccaacccgttcaacacgctggtaccgttcttcccgaacgtggacaccacgcccgcctaccttgaggacaccaccacccgcgtgacgtccgagaacttctactgggccaaccgcatcatcgccgccctgtgcgatggcgccttccgctccacgtccaacgccgttgagcgctaccaggagaagaccggtgcgatgggccaccgcttggtcgccgccaccgacgagcagatcgcccgtctgggcttgacggccgccgaggaagccgcgcagtccgccgctgaagaggaatttgaggccgacaacgtggatggcgatgtgcagccgatgaccccggacgagaccatcgccgcgctgcgtaaccccgaggtgcgcgagattctcgccgccgccaaccagaccatggccgaccagctcaaggaggaaaccgaaaagctgcttgactccgtgctgtacactcgcagcatggaaatgaagaacggcttccacatgtccgacttttagtccgtattgagctcccctccctgaggggagctgaccgcgaagcggactgagggggctaagccgttaagaaaacagcccagtgggctgtttttaggcgaagaggcggcggcagccgccccaccaccccgccccaccatcatcaacacagaaaggctttatatgaccaccatcgaagacttcaccagccagtacggtctggtccagaagatcgacgcattcggctacatgaagtacctggccgagaaccctgacgccccgcgcaagcacggcaaggtggtgctcgtcaccgccgatacgccgctcaaggcatcgcgcggagaaggcaagaccaccacgaccatcgccctgatcgacgccctgaacaaacgcggcattgatgcgaccgccgtgctgcgtcagccgagcatgggcatcaccgcagccggttccaagggcggtgcctccggcggcggcaaggcttccctgacccaccccgagctcatcgactggggtctgtgcggcgaaatgggcgccatcgaggcggcccagaacctgctcgtctccttcgccgagaaggccatcgatgacggcaagctcgacacgattctcgtgccgcgtgtctccgaggtgccgagccgctccctgcgctccaccgccgtggactacggcaagggcaacgtgccggagaaggtcgtgctcacgccgacctgcgagctcatgcagattgtggtgctctcccgctcgatggacgagatttccgagcgcgtgtccaagatgatcgccggcaccaaggacggcaaggccgtgaccttcggcgaattcgtggacctgtggcgtatcactggcattctggccgacgcggtcaagccggccaagaccgagaccgtaaacggctccccggtctacgtgcacggtggcccgttcgccaacgtgtccatcggcattccgacgctggtctccgtggaaatggcctgcgccctgcatgacgtggtcatcgtcgaggccggttacggcaccgacgccggcgcccagaagtggc

SEQ ID NO: 2 - protein, 533 aa, Bifidobacterium longum
MACTTILVGKDASYDGSTIIARNEDSANGEFNPKRFIVVKPEEQPREYKSVISHLTITLPDDPLQYTAVPNADLKEGIWGEAGVNEANVAMSATETLTTNERVLGADPFVEYTPAKGDEPEVPGGIGEEDFLTIVLPYVKTAREGVQRLGALLEEFGTYEMNGVAFSDSNEIWWLETVGGHHWIAKRVPDEAYVTMPNQLGIDEFDLEDALGDQEAHMCSEDLAEFIETNHLDLAVENTTPFNPRDAFGSHSDSDHVYNTPRAWYMQRFLNPYDEVWDGPDADHKPTSDDIPWARQPERKVTIEDIKYVLSSHYQGTPFDPYGQLGDERTRHMYRTIGINRQSQLAVMQIRPYRPQASRAIQWMAYGSNPFNTLVPFFPNVDTTPAYLEDTTTRVTSENFYWANRIIAALCDGAFRSTSNAVERYQEKTGAMGHRLVAATDEQIARLGLTAAEEAAQSAAEEEFEADNVDGDVQPMTPDETIAALRNPEVREILAAANQTMADQLKEETEKLLDSVLYTRSMEMKNGFHMSDF

SEQ ID NO: 3 - protein, 9 aa, Artificial Sequence
  Syntheticaly generated peptide
HLDLAVENT

SEQ ID NO: 4 - DNA, 21 bp, Artificial Sequence
  Primer
aaaaccgattggcaactcggg

SEQ ID NO: 5 - DNA, 19 bp, Artificial Sequence
  Primer
gccacttctgggcgccggc

SEQ ID NO: 6 - DNA, 26 bp, Artificial Sequence
  Primer
atggcctgcaccacgattctggtagg

SEQ ID NO: 7 - DNA, 25 bp, Artificial Sequence
  Primer
aaagtcggacatgtggaagccgttc